(19) United States
(12) Patent Application Publication
(10) Pub. No.: US 2024/0000442 A1
(43) Pub. Date: Jan. 4, 2024

(54) MINIMALLY INVASIVE SKIN SAMPLE COLLECTION APPARATUS

(71) Applicant: Stefan MAZY, New South Wales (AU)

(72) Inventor: Stefan MAZY, New South Wales (AU)

(73) Assignee: DermR Health Solutions Pty Ltd, New South Wales (AU)

(21) Appl. No.: 18/039,576

(22) PCT Filed: Dec. 1, 2021
(86) PCT No.: PCT/AU2021/051431

§ 371 Date: May 31, 2023

(30) Foreign Application Priority Data
Dec. 4, 2020 (AU) 2020904483

(51) Int. Cl. A61B 10/02

(52) U.S. Cl. CPC A61B 10/0233; A61B 2010/0225; A61B 2560/0412

(57) ABSTRACT

A skin sample collection apparatus comprises a sampling patch having an array of only between 4 and 25 epidermis piercing micro-needles on a face surface thereof, thereby able to yield adequate sample whilst significantly reducing invasiveness (causing pain and discomfort) and reducing the force required for the micro-needles to adequately penetrate the epidermis.

### FIELD OF THE INVENTION

This invention relates generally to minimally invasive micro-needle array skin sample devices, and more particularly, to a micro-needle array skin sample device having a less invasive micro-needle density to yield ratio to reduce discomfort, and also methods of production thereof.

### BACKGROUND

Various forms of micro-needle array skin sample in patches including that which is disclosed in US 2017/0145489 A1 (MINDERA CORPORATION) 25 May 2017 which a device containing an array of microneedles to which are attached probes specific for one or more biomarkers of interest.

US 2020/0229803 A1 (GE HEALTHCARE UK LIMITED) 23 Jul. 2020 discloses a similar device for obtaining a skin sample which has an array of micro-needles arranged on a base plate.

US 2003/0036710 A1 (MATRIANO et al.) 20 Feb. 2003 discloses a similar device for collecting nucleic acid on surfaces of the microprojections and/or in a separate nucleic acid collection reservoir.

### SUMMARY OF THE DISCLOSURE

Whereas the above prior art micro-needle sampling devices are less invasive than conventional skin biopsies, they can yet be painful or at least uncomfortable when penetrating the skin.

As such, there is provided herein minimally invasive skin sample collection apparatus specifically suited for home diagnostic use and mail in of samples for analysis.

The apparatus comprises a sampling patch having array of micro-needles on a face surface thereof.

These micro-needles pierce through the outermost layer of the skin and into the underlying epidermis to collect living skin cell samples, including DNA, RNA, or other polynucleic acid material found in the nucleii and/or mitochondria of cells.

Whereas prior art devices employ high concentrations of micro-needles to increase sample yield, our experimentation unexpectedly found that a patch comprising a low micro-needle density of only between 4 and 25 micro-needles, preferably an array of 9 micro-needles, is able to yield adequate sample (as is evident from conventional skin biopsy baseline data being highly correlated with that obtained using the present sampling patch comprising only nine micro-needles as shown in FIG. 9) whilst significantly reducing invasiveness (causing pain and discomfort) and force required for the micro-needles to adequately treat the epidermis.

The apparatus further comprises a sample container into which the skin sample containing patch is inserted. The sample container is relatively small and robust and has a tightfitting lid suitable for mailing in of samples for analysis, such as using Polymerase chain reaction (PCR) or quantitative real-time PCR (qRT-PCR) techniques.

The sample container comprises a buffer solution for preserving the sample during mailing. We further found at a buffer volume of less than 500 μL, preferably approximately 200 μL is suitable to preserve and approximately 1 cm² sampling patch without overly dilating the sample.

The sampling patch is sized either to fit within the container or is flexibly bendable to fit within the container.

The sampling patch preferably comprises an adhesive surface surrounding the micro-needles to additionally sample skin surface microbiome. The adhesive surface preferably surrounds a micro-needle base plate to avoid interfering with barbs thereof. The entire patch may be inserted into the sample container, thereby comprising both transdermal samples collected by the micro-needles and skin surface microbiome samples sampled by the adhesive surface.

The needles are preferably moulded from polymer and may comprise a cross-sectional profile continuously diminishing towards a barbed edge so that the needles can be de-moulded without damaging the barbed edge. These moulded needles can be subsequently adhered perpendicularly to the base plate.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a top plan view of a minimally invasive skin sample collection patch in accordance with an embodiment;

FIGS. 2 and 3 illustrate utilisation of the patch for collection of skin sample;

FIGS. 4 and 5 show a sample container for the sampling patch;

FIG. 6 shows exemplary dimensions of the sampling patch;

FIG. 7 shows a side elevation view of a microneedle in accordance with an embodiment;

FIG. 8 shows a longitudinal cross-sectional profile of a needle being the moulded from a mould; and

FIG. 9 shows a gene expression correlation of samples obtained from a conventional invasive skin biopsy as compared to samples obtained from the present sampling patch.

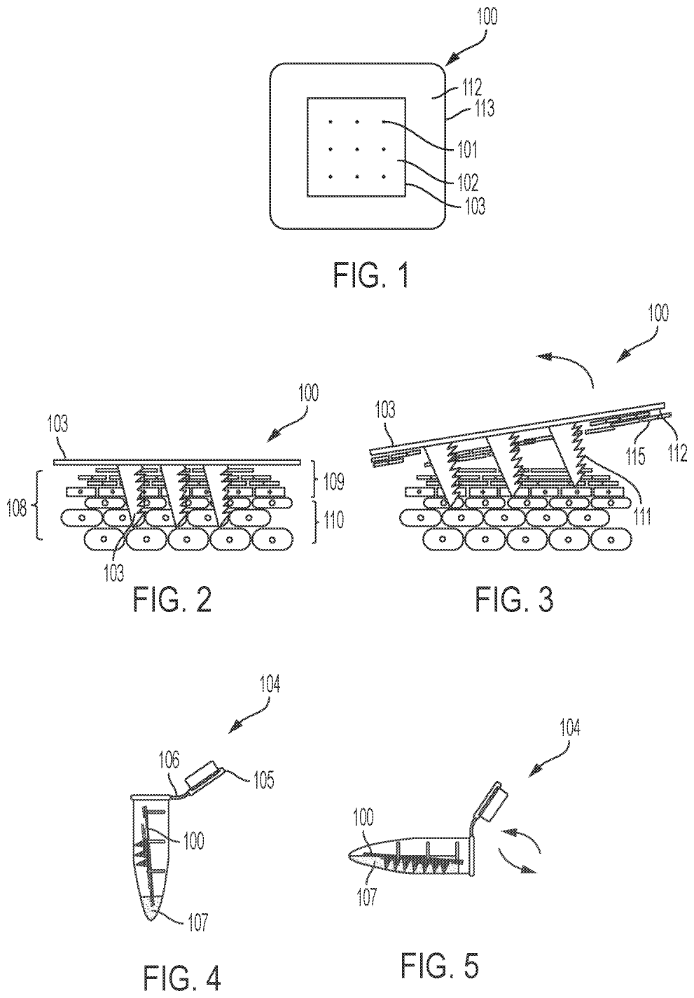

### DESCRIPTION OF EMBODIMENTS

FIG. 1 shows a sampling patch 100 comprising an array of tiny micro-needles 101 on a face surface 102 thereof. The micro-needles 101 preferably pierce the skin to depth of between about 25 μm to 400 μm.

The patch 100 comprises only between 4 and 25 micro-needles 101, preferably between 4 and 16 micro-needles and further preferably 9 micro-needles in the 3×3 array shown.

The patch 100 may comprise a base plate 103 which may be plastic and flexible.

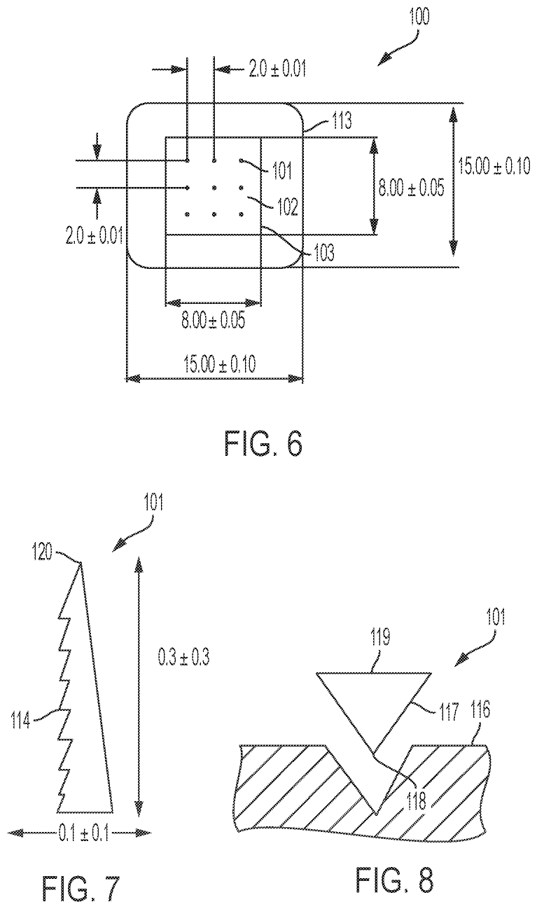

Exemplary dimensions are given in FIG. 6 wherein the base plate 103 may be approximately 8 mm² and wherein the micro-needles 101 are spaced apart more than 1.5 mm from each other, preferably approximately 2 mm apart.

The apparatus further comprises a sample container 104 for the sampling patch 100. The container 104 may be made of plastic and may be small enough to be mailed.

The container 104 may be generally cylindrical having a tapered distal end and an opening enclosed by a tightfitting watertight lid 105 held by a living hinge 106. The container 104 contains buffer solution 107 therein.

FIG. 3 illustrates the application of the patch 100 to the skin 108 wherein the micro-needles 101 penetrate the epidermis 109 into the dermis 110.

FIG. 3 illustrates the removal of the patch 100 wherein the needles 103 comprise subsurface dermis sample 111.

The entire patch 100 is then placed within the container 104 and, as shown in FIG. 5, the container 104 may be rotated to coat the patch 100 with the buffer solution 107.

In a preferred embodiment, the container 104 comprises less than 500 μL of buffer solution, preferably approximately 200 μL for an approximately 1 cm² patch 100. This volume was found to be sufficient to coat the face surface 102 of the patch 100 without over dilution of the sample 111.

The base plate 103 may be sized so as to be able to fit within the interior of the container 104.

Alternatively, the base plate 103 may be wider than the interior of the container 104 wherein the display 103 can bend to fit within the interior of the container 104. In this regard, the base plate 103 may comprise plastic.

In a preferred embodiment, the patch 100 exposes an adhesive surface 112. The adhesive surface 112 may surround the base plate 103. The adhesive surface 112 may be provided by applying an adhesive sheet 113 to a rear of the base plate 103. As shown in FIG. 6, the adhesive sheet 113 may be approximately 15 mm².

The adhesive surface 112 may further hold the base plate 103 to the skin so that the sampling patch 100 may be worn for a period to obtain adequate sample.

Preferably, the patch 100 is devoid of adhesive between the micro-needles 101 so as not to interfere with the barbs 114 thereof.

As is shown in FIG. 3, the adhesive surface 112 may collect epidermis sample 115. As such, the entire patch 100 comprising both the dermis sample 111 and the epidermis sample 112 are inserted into the container 104.

The patch 100 may be sized so that the adhesive sheet 113 fits within the container 104. Alternatively, the adhesive sheet 113 may be wider than the interior of the container and the base plate 103 may be narrower than the interior of the container 104. As such, edges of the adhesive sheet 113 can be folded inward to fit within the container 104.

In further embodiments, for especially small containers, both the adhesive sheet 113 and the base plate 103 are wider than the interior of the container but wherein both can be bent or folded to fit within the container 104.

The micro-needles 103 are preferably cast-in moulded from polymer. FIG. 8 shows wherein a micro-needle 101 is poured into a mould 116, allowed to set and then removed sideways from the mould 116 without damaging the micro-needle 110.

In this regard, the micro-needle 103 may comprise a cross-sectional profile along the length thereof and perpendicular to a longitudinal axis thereof which continuously diminishes towards a barbed edge 118, the barbed edge 118 comprising the barbs 114 shown in FIG. 7.

As shown in FIG. 8, the micro-needle 101 may be generally triangular in cross-section and may comprise a planar rear surface 119.

The barbs 114 may locate along the barbed edge 118 only and the micro-needle 101 may narrow towards a sharpened tip 120.

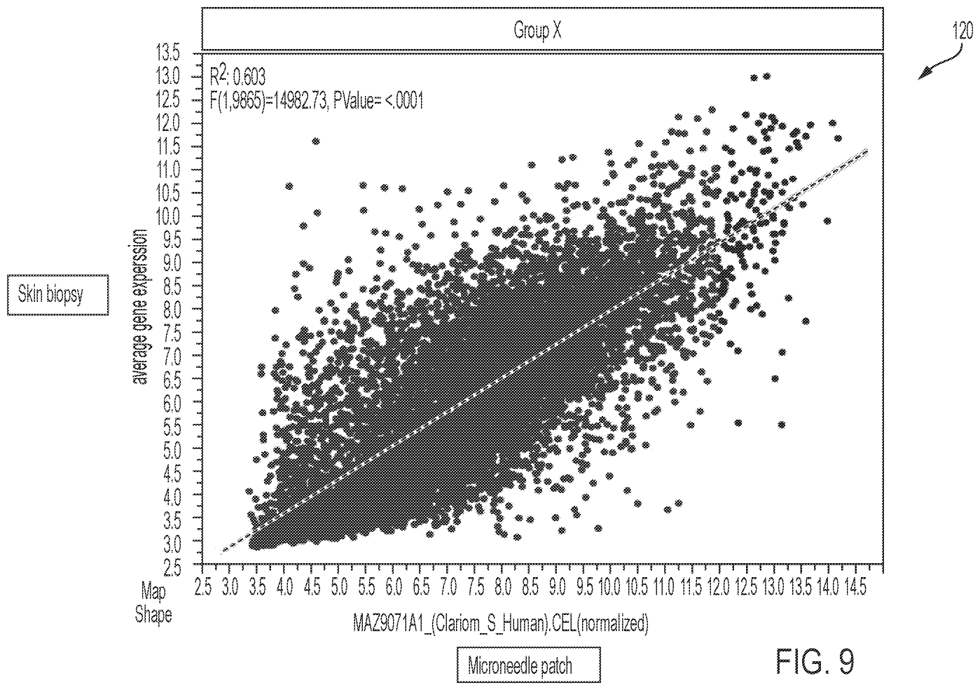

FIG. 9 shows a gene expression correlation of samples obtained from a conventional invasive skin biopsy (shown on the Y-axis) as compared to samples obtained from the present sampling patch 100.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

The term “approximately” or similar as used herein should be construed as being within 10% of the value stated unless otherwise indicated.

1. A skin sample collection apparatus comprising a sampling patch having an array of epidermis piercing micro-needles on a face surface thereof, wherein the number of micro-needles is in a range having:
a lower bound of more than 4; and
an upper bound of less than 25.

2. The apparatus as claimed in claim 1, wherein the upper bound is less than 16.

3. The apparatus as claimed in claim 1, wherein the device has only 9 micro-needles.

4. The apparatus as claimed in claim 1, wherein the micro-needles are spaced apart from each other by more than 1.5 mm.

5. The apparatus as claimed in claim 1, wherein the micro-needles are spaced apart from each other by no less than 2 mm.

6. The apparatus as claimed in claim 1, further comprising a sample container for the patch, the container comprising buffer solution.

7. The apparatus as claimed in claim 6, wherein the patch is 1 cm² and the container comprises less than 500 μl of buffer solution.

8. The apparatus as claimed in claim 7, wherein the container comprises no more than 200 μl of buffer solution.

9. The apparatus as claimed in claim 6, wherein the patch comprises a base plate backing the micro-needles and wherein the base plate fits within the container.

10. The apparatus as claimed in claim 6, wherein the patch comprises a base plate backing the micro-needles, wherein the base plate is flexible and wherein the plate can flexibly bend to fit within the container.

11. The apparatus as claimed in claim 1, wherein the patch exposes an adhesive surface to collect a skin surface sample in addition to a subsurface skin sample collected by the micro-needles.

12. The apparatus as claimed in claim 11, wherein the patch comprises a base plate backing the micro-needles and wherein the patch further comprises an adhesive sheet applied to a rear of the base plate, the adhesive sheet been larger than the base plate to expose the adhesive surface.

13. The apparatus as claimed in claim 11, wherein the adhesive surface surrounds the micro-needles and wherein the face surface is not adhesive between the micro-needles.

14. The apparatus as claimed in claim 1, wherein each needle has cross sectional profile along a length thereof, the profile being perpendicular to a longitudinal axis and continuously diminishing towards a barbed edge.

15. The apparatus as claimed in claim 14, wherein the cross sectional profile is generally triangular.

16. The apparatus as claimed in claim 14, wherein the micro-needles comprise a polymer.

17. A method of collecting a skin sample using the apparatus as claimed in claim 6, the method comprising applying the sampling patch to skin to collect a skin sample with the micro-needles and placing the patch in the container to be covered by the buffer solution.

18. The method as claimed in claim 17, wherein the patch exposes an adhesive surface to collect a skin surface sample in addition to a subsurface skin sample collected by the micro-needles; wherein the skin surface sample collected by the adhesive surface and the subsurface skin sample collected by the micro-needles is placed in the container.

19. A method of manufacturing a micro-needle for the apparatus as claimed in claim 14, the method comprising pouring a polymer into a mould, allowing the polymer to set to form the micro-needle and removing the micro-needle from the mould.

20. A method as claimed in claim 19, wherein the micro-needle has cross sectional profile along a length thereof, the profile being perpendicular to a longitudinal axis and continuously diminishing towards a barbed edge.
